(19) United States
(12) Patent Application Publication
(10) Pub. No.: US 2021/0196947 A1
(43) Pub. Date: Jul. 1, 2021

(54) HEALTHCARE DEVICE USING SMART GARMENT

(71) Applicant: CELLOGIN INC., Wonju-si (KR)

(72) Inventors: Seung Kwan CHO, Goyang-si (KR); Seong Guk Kim, Wonju-si (KR); Chang Yong Ko, Wonju-si (KR); Yeon Sook Weon, Goyang-si (KR); Han Sung Kim, Wonju-si (KR); Tae Min Shin, Wonju-si (KR)

(21) Appl. No.: 16/997,961

(22) Filed: Aug. 20, 2020

(30) Foreign Application Priority Data
Dec. 30, 2019 (KR) 10-2019-0178547

(51) Int. Cl. A61N 1/04; A61N 1/36; A61B 5/053; G16H 40/63

(52) U.S. Cl. CPC A61N 1/0484; A61N 1/36034; G16H 40/63; A61B 5/0537; A61N 1/0408

(57) ABSTRACT

According to an embodiment, a healthcare device using a smart garment comprises a conductive fabric coupled to an inner side of the smart garment and a main body detachably provided to the smart garment, the main body providing micro-current stimulation or low-frequency stimulation via the conductive fabric, and providing a measurement voltage via the conductive fabric to monitor a user's body fat.

### CROSS-REFERENCE TO RELATED APPLICATION(S)

This application is based on and claims priority under 35 U.S.C. 119 to Korean Patent Application No. 10-2019-0178547, filed on Dec. 30, 2019, in the Korean Intellectual Property Office, the disclosure of which is herein incorporated by reference in its entirety.

### TECHNICAL FIELD

The disclosure relates to a healthcare device and a method for managing body fat by the same.

### DESCRIPTION OF RELATED ART

As interest in health and beauty increases, various efforts are being made to maintain and manage weight.

In particular, various products that can manage body fat are coming on the market, and these products may measure body fat or provide an effect of reducing body fat.

Research on such products is actively being conducted by various companies or research groups.

Body fat is divided into subcutaneous fat and visceral fat according to where they are present.

Typically, visceral fat is located in the deep part of the body.

Subcutaneous fat and visceral fat have similar properties, but have different metabolism characteristics due to differences in sites.

Visceral fat secretes cytokines, an inflammatory protein, causing inflammation and reducing the effectiveness of insulin.

As a result, blood sugar and triglycerides increase, causing many adult diseases.

However, since it is located around large blood vessels such as the portal vein, visceral fat may easily be consumed.

Subcutaneous fat is less harmful than visceral fat, but is not easily reduced due to high energy accumulation.

However, since it has a lot of influence on the body shape, excessive accumulation of subcutaneous fat is not good in terms of beauty and reduces mobility a lot, and ultimately is not beneficial to health.

Therefore, a need exists for reducing both subcutaneous fat and visceral fat so as to achieve the purposes of beauty and health.

Korean Patent No. 10-1172711 discloses a belt-type obesity management device that measures abdominal impedance through a plurality of first electrodes attached to a belt surrounding the abdomen, and based on the impedance, obtains the ratio of the amount of subcutaneous fat to the amount of visceral fat, and mixes and provides a first stimulation pulse to stimulate the abdominal muscles to promote the reduction of subcutaneous fat and a second stimulation pulse to stimulate the abdominal muscles to promote the reduction of visceral fat.

However, an actual test result reveals that the first stimulation pulse and the second stimulation pulse do not have a significant effect on body fat reduction.

As the electrodes are attached in the belt type, the site where body fat is reduced is very limited.

Therefore, according to the disclosure, there is provided a healthcare device capable of efficiently reducing both subcutaneous fat and visceral fat and a method for managing body fat using the healthcare device.

### SUMMARY

According to an embodiment, there is provided a healthcare device using a smart garment, which may reduce both subcutaneous fat and visceral fat and measure and provide the state of the body fat.

However, the objects of the embodiments are not limited thereto, and other objects may also be present.

According to an embodiment, a healthcare device using a smart garment, the healthcare device comprises a conductive fabric coupled to an inner side of the smart garment, and a main body detachably provided to the smart garment, the main body providing micro-current stimulation or low-frequency stimulation via the conductive fabric, and providing a measurement voltage via the conductive fabric to monitor a user's body fat.

The conductive fabric may include a stimulation providing electrode attached to an inner side of a front part of the smart garment, in a position corresponding to the user's abdomen, and a monitoring electrode attached to the inner side of the front part of the smart garment, in a side of the user's body. The main body is electrically connected with the stimulation providing electrode and the monitoring electrode, provides the micro-current stimulation or the low-frequency stimulation via the stimulation providing electrode, and measures the body fat based on a current measured via the stimulation providing electrode according to the measurement voltage applied via the monitoring electrode.

The stimulation providing electrode includes a first stimulation providing electrode and a second stimulation providing electrode disposed to face each other. The monitoring electrode includes a first monitoring electrode and a second monitoring electrode disposed apart from each other to face each other, outside the stimulation providing electrode. A first groove and a second groove, which have a predetermined length, are formed in the first stimulation providing electrode and the second stimulation providing electrode, respectively. A first coupler and a second coupler connected with the main body via a wire are formed in the first monitoring electrode and the second monitoring electrode, respectively.

The main body may include a lower main body inserted to an inside of the smart garment through the first groove and then sticking out through the second groove to an outside of the smart garment, and an upper main body providing the micro-current stimulation and the low-frequency stimulation, selectively coupled with the lower main body via a coupling means, and exposed to the outside of the smart garment. The lower main body includes a first electrode terminal disposed to contact the first stimulation providing electrode and a second electrode terminal disposed to contact the second stimulation providing electrode. The upper main body includes a first supply electrode and a second supply electrode, the first supply electrode and the second supply electrode contacting the first electrode terminal and the second electrode terminal, respectively, to supply electrical stimulation in a coupled state of the upper main body and the lower main body. The upper main body includes a third supply electrode coupled with the first coupler via a first wire and a fourth supply electrode coupled with the second coupler via a second wire.

A first protruding electrode protruding upwards from a base surface is coupled to the first electrode terminal, and a second protruding electrode protruding upwards from the base surface is coupled to the second electrode terminal. A first insertion hole to which the first protruding electrode is inserted is formed in the first supply electrode, and a second insertion hole to which the second protruding electrode is inserted is formed in the second supply electrode. When the upper main body and the lower main body are coupled together, the first protruding electrode is inserted through the first groove to the first insertion hole, and the second protruding electrode is inserted through the second groove to the second insertion hole.

The main body includes a display for displaying a result of measurement of the body fat.

The main body includes a wireless communication module for transmitting a result of measurement of the body fat to an external computing or a smart terminal.

The main body may include a battery, a stimulation providing unit providing the micro-current stimulation, the low-frequency stimulation, or the measurement voltage based on power from the battery, and a controller controlling an operation of the stimulation providing unit and performing the body fat measurement. The controller is operated in a normal mode in which the micro-current stimulation and the body fat measurement are repetitively performed according to the user's setting or in an intensive care mode in which the micro-current stimulation and the low-frequency stimulation and the body fat measurement are repetitively performed.

The controller provides the micro-current stimulation via the stimulation providing electrode for a predetermined time period in the normal mode, applies the measurement voltage via the monitoring electrode, measures a current via the stimulation providing electrode, and measures the body fat based on an impedance value calculated based on the measured current and a database in which impedance values and fat body amounts are matched and stored.

The controller sequentially provides the micro-current stimulation or the low-frequency stimulation via the stimulation providing electrode, according to a predetermined order during a predetermined time period in the intensive care mode, applies the measurement voltage via the monitoring electrode, measures a current via the stimulation providing electrode, and measures the body fat based on an impedance value calculated based on the measured current and a database in which impedance values and fat body amounts are matched and stored.

According to an embodiment, a method for managing body fat by a healthcare device using a smart garment comprises providing micro-current stimulation or low-frequency stimulation via a stimulation providing electrode attached to an inner side of the smart garment, applying a measurement voltage via a monitoring electrode attached to an inner side of a front part of the smart garment, and measuring the body fat based on a current measured via the stimulation providing electrode. The stimulation providing electrode and the monitoring electrode are formed of a conductive fabric with a predetermined area.

A controller of the healthcare device is operated in a normal mode in which the micro-current stimulation and the body fat measurement are repetitively performed according to the user's setting or in an intensive care mode in which the micro-current stimulation and the low-frequency stimulation and the body fat measurement are repetitively performed.

The controller provides the micro-current stimulation via the stimulation providing electrode for a predetermined time period in the normal mode, applies the measurement voltage via the monitoring electrode, measures a current via the stimulation providing electrode, and measures the body fat based on an impedance value calculated based on the measured current and a database in which impedance values and fat body amounts are matched and stored.

The controller sequentially provides the micro-current stimulation or the low-frequency stimulation via the stimulation providing electrode, according to a predetermined order during a predetermined time period in the intensive care mode, applies the measurement voltage via the monitoring electrode, measures a current via the stimulation providing electrode, and measures the body fat based on an impedance value calculated based on the measured current and a database in which impedance values and fat body amounts are matched and stored.

The controller provides the low-frequency stimulation via the stimulation providing electrode during a predetermined time period in the intensive care mode, applies the measurement voltage via the monitoring electrode, measures a current via the stimulation providing electrode, and measures the body fat based on an impedance value calculated based on the measured current and a database in which impedance values and fat body amounts are matched and stored.

According to the embodiments of the disclosure, the healthcare device using a smart garment may provide low-frequency stimulation as well as micro-current stimulation to thereby efficiently reduce both subcutaneous fat and visceral fat.

In particular, the healthcare device may induce a reduction in subcutaneous fat via micro-current stimulation and then stimulate both subcutaneous fat and visceral fat via low-frequency stimulation which is highly penetrative.

Further, electrical stimulation may be applied via the stimulation providing electrodes formed of conductive fabric, and the electrical signal generated from the human body may be measured, and based on the measured electrical signal, body fat may be measured in real-time.

Further, various pieces or types of biometric information, e.g., changes in heartrate, may be measured based on the measured electrical signal.

### BRIEF DESCRIPTION OF THE DRAWINGS

A more complete appreciation of the disclosure and many of the attendant aspects thereof will be readily obtained as the same becomes better understood by reference to the following detailed description when considered in connection with the accompanying drawings, wherein:

FIG. 1 is a view illustrating a healthcare device using a smart garment according to an embodiment of the disclosure;

FIG. 2 is a view illustrating a configuration of a smart garment according to an embodiment of the disclosure;

FIG. 3 is a view illustrating a main body of a healthcare device according to an embodiment of the disclosure;

FIG. 4 is a block diagram illustrating a configuration of a main body of a healthcare device according to an embodiment of the disclosure;

FIG. 5 is a view illustrating a configuration of a lower main body of a healthcare device according to an embodiment of the disclosure;

FIG. 6 is a view illustrating a configuration of a lower main body of a healthcare device according to an embodiment of the disclosure;

FIG. 7 is a view illustrating a coupled state of a smart garment and a main body according to an embodiment of the disclosure;

FIG. 8 is a view illustrating a coupled state of a smart garment and a main body according to an embodiment of the disclosure;

FIG. 9 is a view illustrating a coupled state of a smart garment and a main body according to an embodiment of the disclosure;

FIG. 10 is a view illustrating a method for measuring body fat using a healthcare device according to an embodiment of the disclosure;

FIG. 11 is a view illustrating a method of operating a healthcare device according to an embodiment of the disclosure; and

FIG. 12 is a circuit diagram illustrating a configuration for constantly providing micro-current stimulation according to an embodiment of the disclosure.

### DETAILED DESCRIPTION

Hereinafter, embodiments of the disclosure are described in detail to be easily practiced by one of ordinary skill in the art to which the disclosure pertains with reference to the accompanying drawings.

However, the disclosure may be implemented in other various forms and is not limited to the embodiments set forth herein.

For clarity of the disclosure, irrelevant parts are removed from the drawings, and similar reference denotations are used to refer to similar elements throughout the specification.

Throughout the specification, when an element is “connected” with another element, the element may be “directly connected” with the other element, or the element may be “electrically connected” with the other element via an intervening element.

Throughout the specification, when one member is positioned “on” another member, the first member may be positioned directly on the second member, or other member(s) may be positioned between the first and second member.

Hereinafter, embodiments of the disclosure are described in detail with reference to the accompanying drawings.

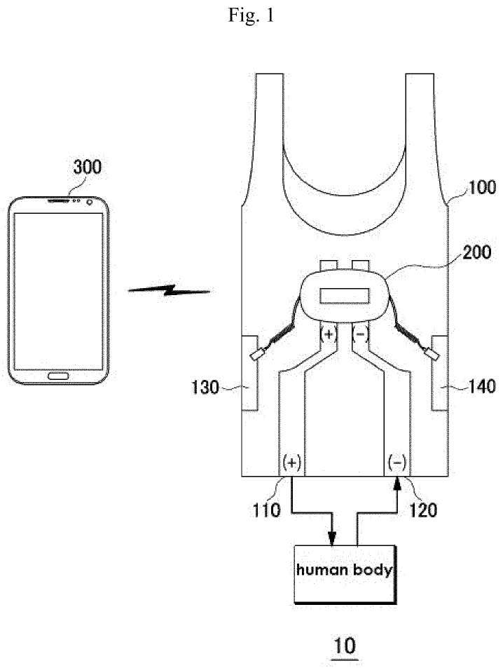

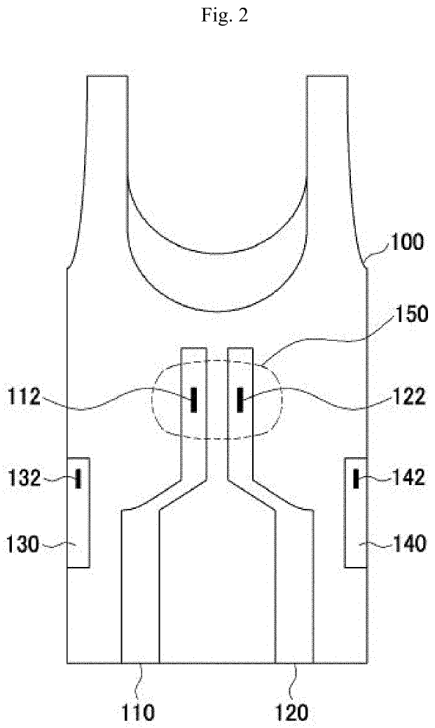

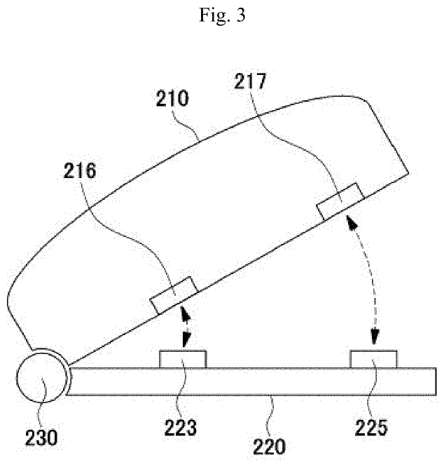

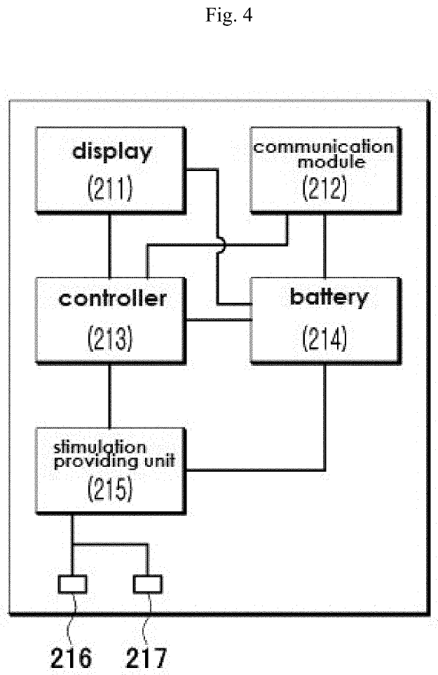

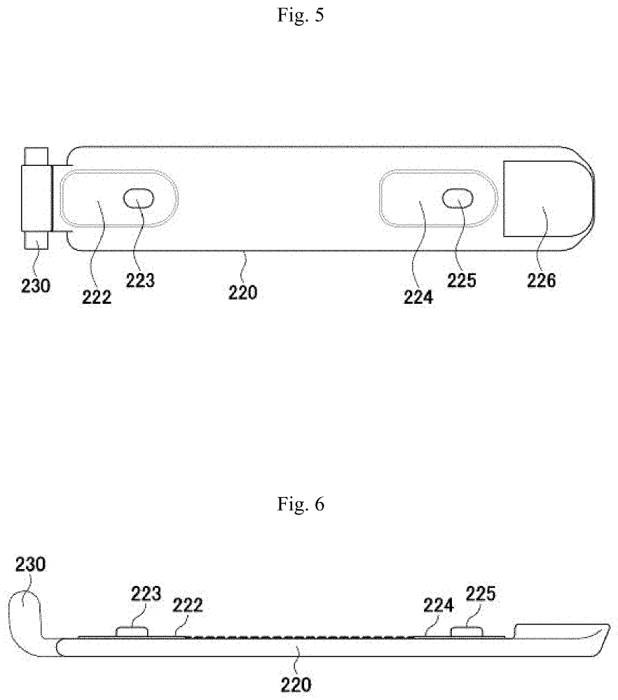

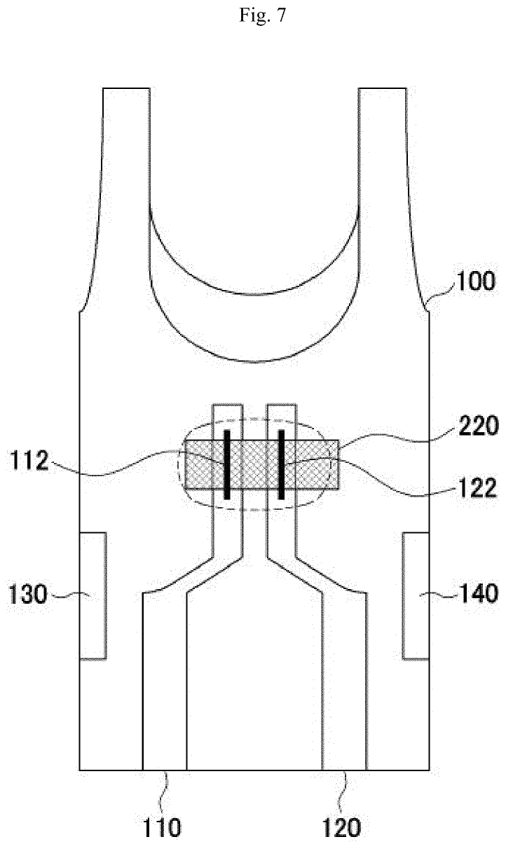

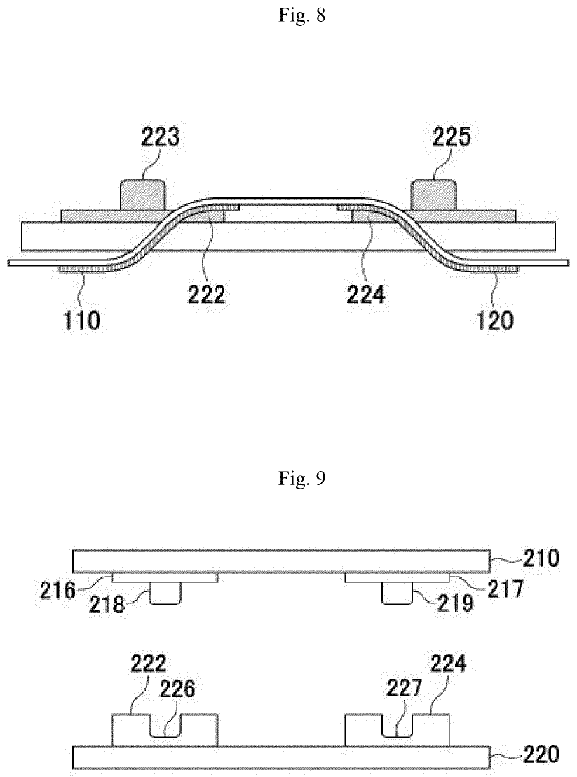

FIG. 1 is a view illustrating a healthcare device using a smart garment according to an embodiment of the disclosure. FIG. 2 is a view illustrating a configuration of a smart garment according to an embodiment of the disclosure. FIG. 3 is a view illustrating a main body of a healthcare device according to an embodiment of the disclosure. FIG. 4 is a block diagram illustrating a configuration of a main body of a healthcare device according to an embodiment of the disclosure. FIG. 5 is a view illustrating a configuration of a lower main body of a healthcare device according to an embodiment of the disclosure. FIG. 6 is a view illustrating a configuration of a lower main body of a healthcare device according to an embodiment of the disclosure. FIG. 7 is a view illustrating a coupled state of a smart garment and a main body according to an embodiment of the disclosure. FIG. 8 is a view illustrating a coupled state of a smart garment and a main body according to an embodiment of the disclosure. FIG. 9 is a view illustrating a coupled state of a smart garment and a main body according to an embodiment of the disclosure.

As shown in the drawings, a healthcare device 10 includes a garment 100, stimulation providing electrodes 110 and 120 attached to an inner side of the garment 100, monitoring electrodes 130 and 140 attached to the inner side of the garment 100, and a main body 200 disposed on a front outer side of the garment 100. The main body 200 is electrically connected with the stimulation providing electrodes 110 and 120 and the monitoring electrodes 130 and 140. The main body 200 provides micro-current stimulation or low-frequency stimulation via the stimulation providing electrodes 110 and 120. The main body 200 provides stimulation and measures body fat for a diet, based on the current measured via the stimulation providing electrodes 110 and 120 according to the measurement voltage applied via the monitoring electrodes 130 and 140.

The garment 100 may be shaped as a common piece of clothing worn on the upper part of the user, but is not limited to a specific shape.

The stimulation providing electrodes 110 and 120, the monitoring electrodes 130 and 140, and the main body 200 are attached to the garment 100, and the garment 100 plays a role to support the attached components.

The stimulation providing electrodes 110 and 120 and the monitoring electrodes 130 and 140 may be conductive fabrics having a predetermined area or size, and may be attached to the inside of the garment 100 by crimping, an adhesive, or stitching or sewing.

The conductive fabric may provide a predetermined degree of elasticity or be elastic or flexible to remain in contact with the user's skin even when the user moves.

Although briefly shown the drawings for ease of description, the stimulation providing electrodes 110 and 120 and the monitoring electrodes 130 and 140 are attached to the inside of the garment 100 to directly contact the user's skin.

Because of having electric conductivity, the stimulation providing electrodes 110 and 120 and the monitoring electrodes 130 and 140 allow for an easier providing of current stimulation to the skin or an easier measurement of current from the skin.

A specific configuration of the conductive fabric is disclosed in, and refers to, Applicant's other Korean patents, e.g., Korean Patent No. 10-1865829, titled “conductive wearable to which micro current is applied,” and Korean Patent No. 10-2023776, titled “diet functional fabric for body fat breakdown and weight loss.”

The stimulation providing electrodes 110 and 120 are attached to the inside of a front part of the garment 100 and include a first stimulation providing electrode 110 and a second stimulation providing electrode 120 which are disposed around the abs or abdomen to face each other.

As shown in FIG. 1, the distance between the first stimulation providing electrode 110 and the second stimulation providing electrode 120 may be reduced toward the upper abdomen to couple the upper parts of the first stimulation providing electrode 110 and the second stimulation providing electrode 120 to the main body 200.

The first stimulation providing electrode 110 and the second stimulation providing electrode 120 are widely disposed around the lower abdomen to effectively provide current stimulation, and the first stimulation providing electrode 110 and the second stimulation providing electrode 120 are narrowly disposed at the top thereof, so that the main body 200, although being small in size, may properly provide current stimulation.

However, embodiments of the disclosure are not limited to such a shape.

As shown in FIG. 2, a first groove 112 (or hole or cut) with a predetermined length is formed in an end of the first stimulation providing electrode 110, and a second groove 122 (or hole or cut) with the same length as the first groove 112 is formed in an end of the second stimulation providing electrode 120, which is positioned to face the first groove 112.

The garment 100 may also have grooves (or holes or cuts), which are substantially the same or similar to the grooves 112 and 122, so that a lower main body 220 described below may be inserted through the grooves, and electrodes 222 and 224 of the lower main body 220 are brought in tight contact with the stimulation providing electrodes 110 and 120.

The monitoring electrodes 130 and 140 are attached to the inside of the front part of the garment 100, corresponding to the side of the sides of the user's body, and the monitoring electrodes 130 and 140 include a first monitoring electrode 130 and a second monitoring electrode 140 spaced apart from each other to face each other.

Preferably, the monitoring electrodes 130 and 140 may be disposed to be spaced farthest apart from each other.

The first monitoring electrode 130 includes a first coupler 132, and the second monitoring electrode 140 includes a second coupler 142. The first coupler 132 and the second coupler 142 are connected with the main body 200 via electric lines or wires.

The first coupler 132 and the second coupler 142 may have substantially similar configurations to the grooves 112 and 122. A portion of a connector positioned at an end of the wire may be inserted, and fastened, to each coupler 132 and 142 so that the connector is electrically connected to each monitoring electrode 130 and 140.

A specific configuration thereof is disclosed in, and refers to, Applicant's Korean patents, e.g., Korean Patent No. 10-1865829, titled “conductive wearable to which micro current is applied.”

As described above, the main body 200 provides micro-current stimulation or low-frequency stimulation through the stimulation providing electrodes 110 and 120.

The main body 200 applies measurement voltage via each monitoring electrode 130 and 140 and measures body fat based on the current measured via each stimulation providing electrode 110 and 120 by the measurement voltage.

The main body 200 includes a display 211, a communication module 212, a controller 213, a battery 214, a stimulation providing unit 215, a first supply electrode 216, and a second supply electrode 217.

The display 211 may output information (e.g., normal mode or incentive care mode) about the operation state of the main body 200 or information about the fat body measured or obtained by the main body 200.

The display 211 is not limited to a specific kind or type.

The communication module 212 transmits the information about the operation state of the main body 200 or the result of measurement of body fat measured or obtained by the main body 200 to an external computing device or a smart terminal.

The communication module 212 may be a wireless communication module and may be implemented as, or include, a Bluetooth or Wi-Fi module or circuit, which may interwork with an external computing device 300 or a smart terminal 300.

Thus, the user may conveniently identify or check information about body fat management state via the external computing device or the smart terminal and accumulate and manage relevant records to thereby identify various pieces, kinds, or types of statistical information.

To that end, the external computing device 300 or the smart terminal 300 may use a separate application that provides the information about the operation state of the main body 200 or the body fat measurement result measured or obtained by the main body 200.

The battery 214 may supply power necessary to drive the main body 200 and may be implemented in various forms.

The stimulation providing unit 215 provides micro-current stimulation or low-frequency stimulation to the stimulation providing electrodes 110 and 120 based on the power from the battery 214, or the stimulation providing unit 215 provides measurement voltage to the monitoring electrodes 130 and 140. The stimulation providing unit 215 may be implemented as, or include, a circuit for performing the functions or operations thereof.

The micro-current stimulation or low-frequency stimulation may be pulsed stimulation having a predetermined frequency and may be provided in the form of, e.g., a square wave or sinusoidal wave.

The micro current or micro-current stimulation refers to a current in μA units, which is less than 1 mA, and the micro current stimulation has a current value of less than 1,000 μA and not less than 1 μA, and the frequency of the micro current is set to not more than 1 kHz and not less than 1 Hz.

The low-frequency stimulation may have a current value of not less than 10 A and less than 100 A, and the frequency of the low-frequency stimulation may be set to not more than 1 Hz and not less than 1 Hz.

Examples of the low-frequency stimulation include electrical muscular stimulation (EMS) and transcutaneous electrical nerve stimulation (TENS).

The stimulation providing unit 215 provides micro-current stimulation or low-frequency stimulation via the first supply electrode 216 and the second supply electrode 217.

The micro-current stimulation and the low-frequency stimulation may be understood simply as differing in the magnitude of current stimulation. However, if the magnitude of current stimulation is varied, many differences may occur in physiological phenomenon in the human body.

Micro-current stimulation is similar in magnitude to biological current, and even when the micro-current stimulation is applied, humans cannot easily feel it, and it affects the cells of the human body.

On the other hand, the low-frequency stimulation has a magnitude enough to be directly felt and affects the tissues, not the cells.

In terms of body fat reduction, micro-current stimulation is a direct stimulation that stimulates the fat cells themselves, and low-frequency stimulation is an indirect stimulation that induces contraction of muscles and, during the course, leads to energy consumption, thereby reducing body fat.

Micro-current stimulation may be safe and effective because it affects the cells, but delivering micro-current stimulation to the deep part of the body is limited as the magnitude of the stimulation is small.

On the other hand, low-frequency stimulation uses the exercise of muscles in the abdomen, so it can affect the entire abdomen including the deep part.

Therefore, a way to reduce both subcutaneous fat and visceral fat may use a combination of the micro-current stimulation and low-frequency stimulation for the subcutaneous fat, which is located at the outside and is not easily broken down, while using the low-frequency stimulation for the visceral fat located in the deep part of the body.

The controller 213 may control the operation of the display 210, the communication module 212, the battery 214, and the stimulation providing unit 215 and, to that end, the controller 213 includes a processor and a memory storing a control program.

The controller 213 may perform a normal mode for providing micro-current stimulation and repetitively measuring body fat according to the user settings.

The controller 213 may perform an incentive care mode for providing micro-current stimulation and low-frequency stimulation and repetitively measuring body fat.

Specific operations using the controller 213 are described below.

As shown in FIG. 3, the main body 200 may be divided into an upper main body 210 and a lower main body 220.

The upper main body 210 provides micro-current stimulation and low-frequency stimulation, is selectively coupled with the lower main body 220 via a coupling means, and is exposed to the outside of the garment 100.

From a layout point of view, the display 211 is preferably disposed on an outer surface of the upper main body 210.

The communication module 212, the controller 213, the battery 214, and the stimulation providing unit 215 may be preferably embedded in the upper main body 210, but some components thereof may be disposed in the lower main body 220.

The lower main body 220 may be inserted into the inside of the garment 100 through the first groove 112 and then stick out to the outside of the garment 100 through the second groove 122.

To that end, the length of the lower main body 220 may be designed to be larger than, at least, the distance between the first groove 112 and the second groove 122, and the width of the lower main body 220 is designed to be smaller than the length of each groove 112 and 122.

The respective first ends of the upper main body 210 and the lower main body 220 may be coupled together via a hinge 230, and the respective second ends, which are opposite to the first ends, of the upper main body 210 and the lower main body 220 may be coupled to each other via a fastening means 226.

The upper main body 210 includes the first supply electrode 216, which contacts a first electrode terminal 222 to supply electrical stimulation in a coupled state of the upper main body 210 and the lower main body 220, and the second supply electrode 217, which contacts a second electrode terminal 224 to supply electrical stimulation in the coupled state of the upper main body 210 and the lower main body 220.

The upper main body 210 includes a third supply electrode (not shown) coupled with the first coupler 132 via a first wire and a fourth supply electrode (not shown) coupled with the second coupler 142 via a second wire.

The lower main body 220 includes the first electrode terminal 222 disposed to contact the first stimulation providing electrode 110 and the second electrode terminal 224 disposed to contact the second stimulation providing electrode 120.

According to an embodiment, a first protruding electrode 223 protruding upwards from a base surface is coupled to the first electrode terminal 222, and a second protruding electrode 225 protruding upwards from the base surface is coupled to the second electrode terminal 224.

The first supply electrode 216 includes a first insertion hole to which the first protruding electrode 223 is inserted, and the second supply electrode 217 includes a second insertion hole to which the second protruding electrode 225 is inserted.

Thus, as shown in FIGS. 7 and 8, when the upper main body 210 and the lower main body 220 are coupled together, the first protruding electrode 223 is inserted through the first groove 112 to the first insertion hole, and the second protruding electrode 225 is inserted through the second groove 122 to the second insertion hole.

By such a coupling structure, the electrically coupled state between the first stimulation providing electrode 110 and the first electrode terminal 222 and the electrically coupled state between the second stimulation providing electrode 120 and the second electrode terminal 224 may be maintained more tightly or securely.

According to another embodiment, as shown in FIG. 9, each protruding electrode may be formed on its corresponding supply electrode, and each insertion hole may be formed in its corresponding electrode terminal.

For example, a first protruding electrode 218 protruding downwards from a base surface may be coupled to the first supply electrode 216, and a second protruding electrode 219 protruding downwards from the base surface may be coupled to the second supply electrode 217.

A first insertion hole 226 may be formed in the first electrode terminal 222 to allow the first protruding electrode 218 to be inserted thereto, and a second insertion hole 227 may be formed in the second electrode terminal 224 to allow the second protruding electrode 219 to be inserted thereto.

Thus, when the upper main body 210 and the lower main body 220 are coupled together, the first protruding electrode 218 may be inserted through the first groove 112 to the first insertion hole 226, and the second protruding electrode 219 may be inserted through the second groove 122 to the second insertion hole 227.

By such a coupling structure, the electrically coupled state between the first stimulation providing electrode 110 and the first electrode terminal 222 and the electrically coupled state between the second stimulation providing electrode 120 and the second electrode terminal 224 may be maintained more tightly or securely.

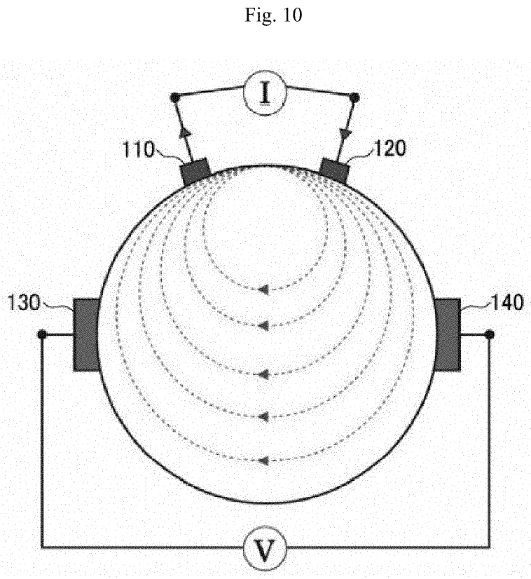

FIG. 10 is a view illustrating a method for measuring body fat using a healthcare device according to an embodiment of the disclosure.

According to an embodiment, body fat is measured using impedance measurement technology.

For example, the impedance of the human body may be calculated by measuring the current flowing through the human body, with a predetermined voltage applied to two opposite ends of the human body, as shown in FIG. 10.

The controller 213 may input the calculated impedance to a previously established database in which impedance values and fat body amounts are matched and obtain the fat body amount matching the calculated impedance.

To that end, the controller 213 applies a predetermined voltage through each monitoring electrode 130 and 140 and measures the current flowing through the user's body via each stimulation providing electrode 110 and 120.

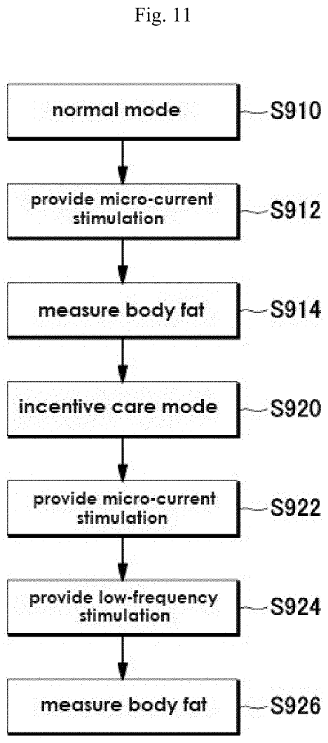

FIG. 11 is a view illustrating a method of operating a healthcare device according to an embodiment of the disclosure.

Referring to FIG. 11, in the normal mode S910, micro-current stimulation is provided through each stimulation providing electrode 110 and 120 during a preset time period (S912).

In this case, the time period during which the micro-current stimulation is provided or the magnitude of the micro-current stimulation may be varied according to embodiments.

Micro-current stimulation does not cause any discomfort to the user in everyday life even when provided to the user all day long.

After the micro-current stimulation is provided, body fat measurement is performed (S914).

To that end, a measurement voltage is applied through the monitoring electrodes 130 and 140, the current is measured through the stimulation providing electrodes 110 and 120, and the body fat is measured based on a previously established or configured database in which impedance values and fat body amounts are matched and stored, and the impedance value calculated based on the measured current.

In the incentive care mode S920, micro-current stimulation and low-frequency stimulation are sequentially provided via the stimulation providing electrodes 110 and 120 according to a predetermined order during a predetermined time period (S922 and S924).

Alternatively, only low-frequency stimulation may be provided during a predetermined time period.

Although an example is illustrated in which micro-current stimulation is provided and then low-frequency stimulation is provided, this does not mean that the steps are limited to the order but rather than each step may be independently performed.

In the intensive care mode S920, various combinations of micro-current stimulation and low-frequency stimulation may be provided.

For example, after micro-current stimulation and then low-frequency stimulation are provided, micro-current stimulation may be provided again, or after low-frequency stimulation and then micro-current stimulation are provided, low-frequency stimulation may be provided again. As such, the incentive care mode may be configured in various manners or fashions or patterns.

Or, the incentive care mode may be configured to provide only low-frequency stimulation during a predetermined time period.

Thereafter, body fat measurement (S926) may be performed, and a specific method thereof is substantially the same as step S914 described above.

The healthcare device of the disclosure provides micro-current stimulation and may adopt a special configuration for providing constant micro current.

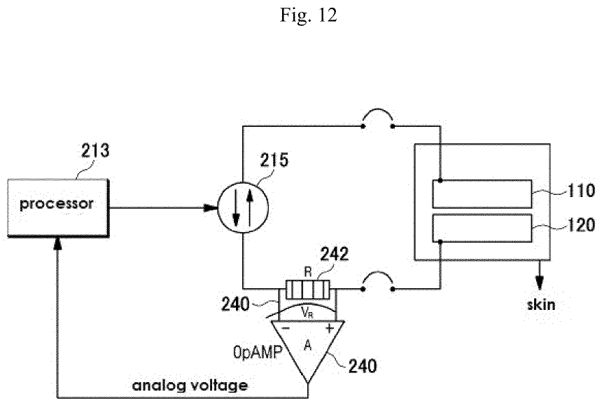

FIG. 12 is a circuit diagram illustrating a configuration for constantly providing micro-current stimulation according to an embodiment of the disclosure.

Since micro-current stimulation is provided to the user's skin for a long time in many cases, it may need to remain at a constant level.

Although the stimulation providing electrodes are formed of conductive fabrics to allow the stimulation providing unit to remain in contact with the skin, the contact may be lost, e.g., if the skin is dry.

Providing micro-current stimulation when the contact to the skin is not secure may be disadvantage in light of power and circuit stability.

According to an embodiment, to detect whether the stimulation providing electrodes 110 and 120 are open or open-circuited, a detection resistor 242 may be added to the closed circuit connecting the stimulation providing unit 215, the first stimulation providing electrode 110, the user's body, and the second stimulation providing electrode 120, and an amplifier 240 is coupled to the detection resistor 242 to amplify the voltage difference between the two ends of the detection resistor 242.

Since micro current flows through the detection resistor 242 in the normal operation, the amplified value of the voltage difference between the two ends of the detection resistor 242 falls within a range of a predetermined value.

However, if the contact between the first stimulation providing electrode 110 or second stimulation providing electrode 120 and the skin is not secure, no or little micro current flows, so that the value output from the amplifier 240 may be smaller than the predetermined value.

The controller 213 controls the operation of the stimulation providing unit 215 according to the value output from the amplifier 240.

In other words, if the output of the amplifier 240 falls within the range of the predetermined value, the controller 213 may allow the stimulation providing unit 215 to maintain the same operation.

However, if the output of the amplifier 240 is smaller than the predetermined value, the controller 213 may allow the stimulation providing unit 215 to temporarily stop operation.

To temporarily stop operation is merely to temporarily stop the stimulation but not to stop the entire operation of the device. Thus, whether the stimulation providing electrodes 110 and 120 are open or open-circuited may be redetected and, if the amplified value between the two ends of the detection resistor 242 falls within the range of the predetermined value, stimulation may be resumed.

According to an embodiment, the method of operating the healthcare device may be implemented in the form of a computer-readable medium including computer-executable commands or instructions such as a program module.

The computer-readable medium may be an available medium that is accessible by a computer. The computer-readable storage medium may include a volatile medium, a non-volatile medium, a separable medium, and/or an inseparable medium.

The computer-readable medium may include a computer storage medium.

The computer storage medium may include a volatile medium, a non-volatile medium, a separable medium, and/or an inseparable medium that is implemented in any method or scheme to store computer-readable commands, data architecture, program modules, or other data or information. All or some of the components or operations of the disclosure may be implemented in or by a computer system having a general-purpose hardware architecture or a dedicated computer, computer system, or device.

Although embodiments of the disclosure have been described with reference to the accompanying drawings, It will be appreciated by one of ordinary skill in the art that the disclosure may be implemented in other various specific forms without changing the essence or technical spirit of the disclosure.

Thus, it should be noted that the above-described embodiments are provided as examples and should not be interpreted as limiting.

Each of the components may be separated into two or more units or modules to perform its function(s) or operation(s), and two or more of the components may be integrated into a single unit or module to perform their functions or operations.

It should be noted that the scope of the disclosure is defined by the appended claims rather than the described description of the embodiments and include all modifications or changes made to the claims or equivalents of the claims.

What is claimed is:

1. A healthcare device using a smart garment, the healthcare device comprising:
a conductive fabric coupled to an inner side of the smart garment; and
a main body detachably provided to the smart garment, the main body providing micro-current stimulation or low-frequency stimulation via the conductive fabric, and providing a measurement voltage via the conductive fabric to monitor a user's body fat.

2. The healthcare device of claim 1, wherein the conductive fabric includes:
a stimulation providing electrode attached to an inner side of a front part of the smart garment, in a position corresponding to the user's abdomen; and
a monitoring electrode attached to the inner side of the front part of the smart garment, in a side of the user's body, and wherein
the main body is electrically connected with the stimulation providing electrode and the monitoring electrode, provides the micro-current stimulation or the low-frequency stimulation via the stimulation providing electrode, and measures the body fat based on a current measured via the stimulation providing electrode according to the measurement voltage applied via the monitoring electrode.

3. The healthcare device of claim 2, wherein
the stimulation providing electrode includes a first stimulation providing electrode and a second stimulation providing electrode disposed to face each other, wherein
the monitoring electrode includes a first monitoring electrode and a second monitoring electrode disposed apart from each other to face each other, outside the stimulation providing electrode, wherein
a first groove and a second groove, which have a predetermined length, are formed in the first stimulation providing electrode and the second stimulation providing electrode, respectively, and wherein
a first coupler and a second coupler connected with the main body via a wire are formed in the first monitoring electrode and the second monitoring electrode, respectively.

4. The healthcare device of claim 3, wherein
the main body includes:
a lower main body inserted to an inside of the smart garment through the first groove and then sticking out through the second groove to an outside of the smart garment; and
an upper main body providing the micro-current stimulation and the low-frequency stimulation, selectively coupled with the lower main body via a coupling means, and exposed to the outside of the smart garment, wherein
the lower main body includes a first electrode terminal disposed to contact the first stimulation providing electrode and a second electrode terminal disposed to contact the second stimulation providing electrode, wherein
the upper main body includes a first supply electrode and a second supply electrode, the first supply electrode and the second supply electrode contacting the first electrode terminal and the second electrode terminal, respectively, to supply electrical stimulation in a coupled state of the upper main body and the lower main body, and wherein
the upper main body includes a third supply electrode coupled with the first coupler via a first wire and a fourth supply electrode coupled with the second coupler via a second wire.

5. The healthcare device of claim 2, wherein
a first protruding electrode protruding upwards from a base surface is coupled to the first electrode terminal, and a second protruding electrode protruding upwards from the base surface is coupled to the second electrode terminal, wherein
a first insertion hole to which the first protruding electrode is inserted is formed in the first supply electrode, and a second insertion hole to which the second protruding electrode is inserted is formed in the second supply electrode, and wherein
when the upper main body and the lower main body are coupled together, the first protruding electrode is inserted through the first groove to the first insertion hole, and the second protruding electrode is inserted through the second groove to the second insertion hole.

6. The healthcare device of claim 1, wherein the main body includes a display for displaying a result of measurement of the body fat.

7. The healthcare device of claim 1, wherein the main body includes a wireless communication module for transmitting a result of measurement of the body fat to an external computing or a smart terminal.

8. The healthcare device of claim 1, wherein the main body includes:
a battery;
a stimulation providing unit providing the micro-current stimulation, the low-frequency stimulation, or the measurement voltage based on power from the battery; and
a controller controlling an operation of the stimulation providing unit and performing the body fat measurement, and wherein
the controller is operated in a normal mode in which the micro-current stimulation and the body fat measurement are repetitively performed according to the user's setting or in an intensive care mode in which the micro-current stimulation and the low-frequency stimulation and the body fat measurement are repetitively performed.

9. The healthcare device of claim 8, wherein
the controller provides the micro-current stimulation via the stimulation providing electrode for a predetermined time period in the normal mode, applies the measurement voltage via the monitoring electrode, measures a current via the stimulation providing electrode, and measures the body fat based on an impedance value calculated based on the measured current and a database in which impedance values and fat body amounts are matched and stored.

10. The healthcare device of claim 8, wherein
the controller sequentially provides the micro-current stimulation or the low-frequency stimulation via the stimulation providing electrode, according to a predetermined order during a predetermined time period in the intensive care mode, applies the measurement voltage via the monitoring electrode, measures a current via the stimulation providing electrode, and measures the body fat based on an impedance value calculated based on the measured current and a database in which impedance values and fat body amounts are matched and stored.

11. A method for managing body fat by a healthcare device using a smart garment, the method comprising:
providing micro-current stimulation or low-frequency stimulation via a stimulation providing electrode attached to an inner side of the smart garment;
applying a measurement voltage via a monitoring electrode attached to an inner side of a front part of the smart garment; and
measuring the body fat based on a current measured via the stimulation providing electrode, wherein
the stimulation providing electrode and the monitoring electrode are formed of a conductive fabric with a predetermined area.

12. The method of claim 10, wherein a controller of the healthcare device is operated in a normal mode in which the micro-current stimulation and the body fat measurement are repetitively performed according to the user's setting or in an intensive care mode in which the micro-current stimulation and the low-frequency stimulation and the body fat measurement are repetitively performed.

13. The method of claim 12, wherein
the controller provides the micro-current stimulation via the stimulation providing electrode for a predetermined time period in the normal mode, applies the measurement voltage via the monitoring electrode, measures a current via the stimulation providing electrode, and measures the body fat based on an impedance value calculated based on the measured current and a database in which impedance values and fat body amounts are matched and stored.

14. The method of claim 12, wherein
the controller sequentially provides the micro-current stimulation or the low-frequency stimulation via the stimulation providing electrode, according to a predetermined order during a predetermined time period in the intensive care mode, applies the measurement voltage via the monitoring electrode, measures a current via the stimulation providing electrode, and measures the body fat based on an impedance value calculated based on the measured current and a database in which impedance values and fat body amounts are matched and stored.

15. The method of claim 12, wherein
the controller provides the low-frequency stimulation via the stimulation providing electrode during a predetermined time period in the intensive care mode, applies the measurement voltage via the monitoring electrode, measures a current via the stimulation providing electrode, and measures the body fat based on an impedance value calculated based on the measured current and a database in which impedance values and fat body amounts are matched and stored.
